(19) United States
(12) Patent Application Publication
(10) Pub. No.: US 2017/0143844 A1
(43) Pub. Date: May 25, 2017

(54) Composition and Methods of Use of Nano Anti-Radical Therapeutics To Inhibit Cancer

(71) Applicant: AntiRadical Therapeutics LLC, Sioux Falls, SD (US)

(72) Inventor: Jen-Chang Hsai, Sioux Falls, SD (US)

(21) Appl. No.: 15/360,264

(22) Filed: Nov. 23, 2016

Related U.S. Application Data
(60) Provisional application No. 62/259,485, filed on Nov. 24, 2015.

(51) Int. Cl. A61K 38/00; A61K 31/445; A61K 31/337

(52) U.S. Cl. CPC A61K 47/48284; A61K 31/445; A61K 31/337

(57) ABSTRACT

This invention relates to the compositions and methods of use of macromolecular nitroxide as a nano anti-radical therapy (NanoART) to compliment current chemotherapy, radiotherapy, immunotherapy, hormonal therapy, biological therapy and surgery in the treatment of cancer. NanoART is unique in that it inhibits all three key steps in carcinogenesis. NanoART inhibits proliferation, progression and metastasis of cancer processes through the free radical based reactive oxygen species pathway. This is a novel comprehensive therapy which has not been addressed by current standard cancer therapies with a single therapeutic agent. NanoART may also exert its anti-cancer activities throughout the body regardless of the location of its administration, and this is a revolutionary new paradigm in medical treatment. As a treatment of carcinogenesis, NanoART has the potential to decrease morbidity, decrease mortality and improve the cure of cancer.

### CROSS REFERENCE TO RELATED APPLICATIONS

This application claims the benefit of the filing date of U.S. Provisional Application Ser. No. 62/259,485, filed on Nov. 25, 2015 and entitled Compositions and Methods of Use of Nano Anti-Radical Therapeutics to Inhibit Cancer, the entire disclosure of which is incorporated herein by reference.

### STATEMENT REGARDING FEDERALLY SPONSORED RESEARCH OR DEVELOPMENT

Not applicable

### JOINT RESEARCH AGREEMENT

Not applicable

### BACKGROUND OF THE INVENTION

Field of the Invention

The present invention relates to compositions and methods of use of macromolecular nitroxide which reduces morbidity and mortality in cancer patients through the inhibition of carcinogenesis including metastasis.

It is desired to provide a multifunctional therapy for cancer in order to improve the survival of cancer patients. Ideally, these functions should reside in a single cancer treatment drug which works through the inhibition of cancer cell proliferation, progression and metastasis associated inhibition of reactive oxygen species (ROS) resulting in the enhancement of blood flow and therapeutic index of standard cancer therapies.

We describe here the development of such a class of macromolecular nitroxide with serum albumin as the macromolecular carrier of the nitroxide, for the reduction of morbidity and mortality in cancer patients.

Generally, the treatment of metastatic cancer aims to slow the growth or spread of the cancer. Treatment should be independent of the type of cancer, where it started, the size and location of the metastasis, and other factors. Typically, metastatic cancer requires systemic therapy, or medications given by mouth or injected into the bloodstream to reach cancer cells throughout the body, such as chemotherapy or hormone therapy. Other treatments may include biological therapy, chemotherapy, radiation therapy and immunotherapy, surgery, or a combination of these. Thus, development of a comprehensive treatment for carcinogenesis to improve the cure of cancer is needed. There is also a basic paradigm in the current standard treatment regime namely that therapeutic agents must be administered orally, intravascularly and retained in the bloodstream (intravascular space) in order to exert ant-cancer activity. The present invention overcomes this limitation.

Hypoxia, a lack of blood flow into the hypoxic core of tumors is the root cause of cancer metastasis and tumor propagation. Lack of blood flow also causes resistance to drug therapy by blocking drug access. Existing cancer therapies lack anti-hypoxia activity through the catalytic vascular superoxide removal activity and do not increase blood flow into the core of tumors. One example of an existing therapy is nab-paclitaxel (also known as ABI-007; nanoparticle albumin-bound paclitaxel). Although nabpaclitaxel is a carrier of the anti-microtubule drug paclitaxel, the bound paclitaxel has lowered bioavailability, due to the lack of blood flow inside the tumor hypoxic core.

There are numerous patents addressing the compositions and methods for the treatment of cancer carcinogenesis. However, to the best of knowledge of the inventor, none of the previously known compositions and methods include the therapeutic efficacy of the present invention for the use of macromolecular nitroxide of the appropriate molecular size as a comprehensive treatment of carcinogenesis and metastasis. Furthermore, none of the previously known compositions and methods include regulating the intracellular, extravascular and intravascular free radical based reactive oxygen species (ROS) activities is the unified therapeutic mechanism in the treatment of carcinogenesis and metastasis as claimed in this invention.

In summary, against this background, the paradigm shift described in this invention on cancer therapy has been demonstrated with a single therapeutic agent i.e. Multiple Catalytic Free Radical Nitroxides carried on a Polynitroxylated Albumin (PNA).

Acronym Listing

EPR Electron paramagnetic Resonance

HER2 Human Epidermal Growth Factor Receptor 2

HNSCC Head Neck Squamous Cell Carcinoma

HPLC High Performance Liquid Chromatography

HSA Human Serum Albumin

IV Intravenous

IT Intrathecal

IP Intra-peritoneal

MCFRN Multiple Catalytic Free Radical Nitroxides

NAT Nano-Antiradical Therapy

NAB Nanoparticle Albumin Bond

PNA Polynitroxylated Albumin

PNH Polynitroxylated Hemoglobin

ROS Reactive Oxygen Species

SCD Sickle Cell Disease

TNBC Triple Negative Breast Cancer

### SUMMARY OF THE INVENTION

This invention relates to our discovery that in pre-clinical in vitro and in vivo model studies that the extra-cellular macromolecular nitroxide described herein strategically localizes throughout all the extravascular and intravascular volume along the path of cancer cell development to block metastasis. Said macromolecular nitroxide targets the reduction of ROS in the cancer carcinogenesis process, thus creating a new cancer therapy referred to herein as Nano-Antiradical Therapy (NAT), and which specifically target the prevention and treatment of metastasis to complement the current cancer therapies. Novel compositions of macromolecular nitroxide and their methods of use are disclosed. In the present invention the polynitroxylated albumin (PNA) localizes within all body fluids following intraperitoneal (IP) injection. Intrathecal (IT) injection of PNA in the cerebral spinal fluid was also found to be safe and effective in preclinical studies in rat model of hemorrhagic stroke in rat. These findings provide significant improvement over current standard cancer therapies that anti-cancer therapies must be administered orally or intravascularly to be retained in the bloodstream or intravascular space. We found the treatment of cancer carcinogenesis using the formulation of our polynitroxylated albumin (PNA) leads to the inhibition of carcinogenesis of cancer and formation of secondary tumors at other locations in the body.

We present results to show that in preclinical mouse model of triple negative breast cancer (TNBC) model, delivery of the PNA to all body compartments can be accomplished through IP injection to achieve the desired therapeutic effect exemplified by targeting metastasis of TNBC cells from the flank tumor to the lung. The present invention, which was originally designed to treat stroke and sickle cell disease (SCD), has completed FDA IND safety studies and in the stroke and sickle cell disease context is essentially free of toxic side effects as compared to the standard cancer therapeutic treatment. This lack of toxicity would contribute both to the quality and longevity of the life span of cancer patients.

We have used IP administration of PNA in this present invention to demonstrate therapeutic efficacies in a TNBC mouse model leading to the inhibition of metastasis, which is a truly remarkable and novel discovery.

While PNA is employed in a preferred embodiment in this invention, polynitroxylated biocompatible starch and synthetic macromolecules of similar molecules weight as albumin or hemoglobin are alternative embodiments that would have similar anti-cancer activities. The present invention advances macromolecular nitroxides to expand their utility in both intravascular and extravascular spaces to intercept the metastasis and carcinogenesis of cancer, for the improvement of cure of cancer.

The anti-carcinogenesis efficacy and mechanism of action of a NAT agents works in both the intravascular and extravascular space. An example is a Multiple Catalytic Free Radical Nitroxides (MCFRN) carried on Human Serum Albumin (HSA), hereinafter referred to as (“MCFRN-HSA”), which is shown to work in the inhibition of cancer carcinogenesis and metastasis in this invention. We presented representative data in this invention to show that MCFRN-HSA as a single molecule, in a dose dependent manner, is capable of inhibition of proliferation, progression and metastasis of in vitro and in vivo TNBC models. Again this invention demonstrated the remarkable accomplishment that these therapeutic activities resides in a single therapeutic agent, MCFRN-HSA. One particularly beneficial aspect of the present invention is the switch away from the focus of PNA and polynitroxylated hemoglobin (PNH) utility in the intravascular space to BOTH the intravascular and extravascular space. The common denominator of these cancer development processes resides in the role of free radical based ROS is also a key to this invention. To date, ROS has been shown to have both pro-tumorigenic and anti-tumorigenic activities in carcinogenesis, therefore it beneficial effect has not been fully realized as part of the current standard cancer therapies. The inventor believes that the particular deficit of the current cancer therapies is the lack of understanding of compartmentalization of the ROS reactions from intracellular to extravascular verses intravascular to extravascular space in carcinogenesis. Therefore, we have employed MCFRN-HSA as an example of NAT to exert its comprehensive therapeutic effects to cover the full carcinogenesis process driven by ROS activities. We have also strategically placed MCFRN-HSA in all extracellular fluid of the body by demonstrating its efficacy in the inhibition of metastasis following IP administration of this drug.

This invention is developed against the background of the successful clinical use of Trastuzumab, a humanized monoclonal antibody directed against human epidermal growth factor receptor 2 (HER2) and nanoparticle albumin-bound paclitaxel (nab-paclitexal). We describe an example of a macromolecular nitroxide, MCFRN-HSA, which has much broader therapeutic activities than Trastuzumab. Trastuzumab is limited for use only in the inhibition of proliferation of HER2 positive breast cancer patients. In addition, MCFRN-HSA or PNA nanoparticle has been prepared and can used as a carrier of paclitaxel with higher therapeutic efficacy than nanpaclitexal. The albumin in nab-paclitaxel is only a carrier for paclitaxel and has no known therapeutic efficacy of its own in reducing hypoxia and metastasis of cancer. Clinical protocols for the use of nab-paclitexal also specify that it must be administered intravenously and retained within the bloodstream. MCFRN-HSA improves on nab-paclitexal and paclitexal by:
(1) removal of the hypoxia in tumors in advanced cancer to block the extravasation of cancer cells leading to metastasis;
(2) enhancement of blood flow into the hypoxic region of the cancer to improve the delivery, bioavailability and efficacy of paclitaxel; and
(3) larger body fluid drug distribution.

Nitroxide labeling of albumin in MCFRN-HSA results in the reduction of superoxide levels in the bloodstream, leading to increased blood flow into the hypoxic core of tumors. This provides both anti-cancer progression and anti-metastasis activities in a single drug.

### DESCRIPTION OF THE DRAWINGS

FIG. 1. Colony forming assays in MCFRN-HSA treated 4T1 cultures. Cells were plated at low density and then treated with the indicated concentration of MCFRN-HSA as a control. Colonies were stained and counted (A) or measured for size (B).

FIG. 2. Effects of MCFRN-HSA on cellular ROS levels. 4T1 cells were treated with MCFRN-HSA as indicated, stained with DCF and analyzed by flow cytometry. Mean fluorescence intensity is shown. Lower fluorescence indicates lower ROS levels.

FIG. 3. Expression of cyclin D1 in MCFRN-HSA treated 4T1 cells. Cultured 4T1 cells were treated with MCFRN-HSA as indicated and then harvested for western blotting.

FIG. 4. Measurement of blood flow in 4T1 tumors in mice. Tumor bearing animals were injected with microbubbles and then with MCFRN-HSA and time of tumor perfusion was measured by ultrasound. Lower time is indicative of faster blood flow through tumor.

FIG. 5. Effect of MCFRN-HSA on lung metastasis in 4T1 breast cancer model. A) Tumor bearing mice were treated with or without MCFRN-HSA and after 3 weeks lungs were harvested for counting lung metastatic nodules. B) Same as (A) except that mice were also treated with doxorubicin. Metastatic nodules were significantly reduces with a P value<0.01.

### DETAILED DESCRIPTION

### Composition

MCFRN-HSA is prepared by covalent labelling of HSA as the macromolecule using chemically reactive nitroxides to produce PNA. This is produced by the reaction of 4-(2-bromoamindoacetyl)-2,2,6,6-tetramethyl-1-piperidinyloxyl (BrAcTPO) and HSA with bromine as the leaving group. This procedure is called polynitroxylation. Each milliliter of VACNO—10% contains 0.1 gm of HSA and 19 milligram of AcTPO covalently attached to the albumin molecules. The nitroxides so attached function as a “caged nitric oxide” (CNO), which serve as solid analogue of gaseous nitric oxide (NO). CNO catalytically dismutate superoxide to: 1) stop the initiation of inflammatory free radical cascade; 2) inhibit hemoglobin neurotoxicity from hemorrhage; 3) restore the vascular endogenous NO to restore blood flow and eliminate ischemia, and; 4) inhibit oxidative stress from reperfusion injury. MCFRN-HSA may also be subsequently formulated as a 120 mm particle complex with a chemotherapeutic drug such as paclitaxel where MCFRN-HSA also acts as a carrier for the hydrophobic drug paclitaxel to increase its bioavailability. MCFRN-HSA formulated with bound paclitaxel is used as a solution or is lyophilized or is formed into 120 nm nanoparticles using a high pressure homogenizer. For lyophilized MCFRN-HSA complexed with paclitaxel, after re-constitution with saline each dose will contain 900 mg of albumin, 100 mg of paclitaxel, and 150 mg nitroxide in 20 ml. MCFRN-HSA is administrated by either IP, IT or intravenous (IV) injection as one example embodiment. In a second preferred embodiment the macromolecule is hemoglobin or other heme-proteins. In a third example embodiment the macromolecule is starch or a synthetic macromolecule of similar molecular weight as HSA. MCFRN-HSA, or MCFRN-PNA/HSA nanoparticle complexed with paclitaxel or other drug is administered by medical specialists into any body IP, IT or IV compartment. In one preferred embodiment MCFRN-HSA is administered by IP injection. In a second preferred embodiment MCFRN-HSA is administered by IV injection.

Hemoglobin, other heme-proteins, starch, or synthetic macromolecules of similar molecular weight as HSA are considered biocompatible colloids.

Polynitroxyl labelling of albumin confers the anti-hypoxic activities of this invention and the increase in blood flow into the hypoxic core of cancer. Binding pacilitaxel to MCFRN-HSA or its nanoparticle as the bound paclitaxel when delivered to tumor cells provides the anti-mitotic and antiprogression cancer drug activities of the drug. MCFRN-HSA complexed with paclitaxel manufactured and used as 120 nanoparticles is to provide an alternative formulation that minimizes extravasation from the bloodstream of albumin monomer. This will increase MCFRN-HSA and paclitaxel levels and their half life in the bloodstream.

In the MCFRN-HSA, the number of nitroxides on the albumin molecule is tested and can be verified to be between 40-60 nitroxides per molecule of HSA. This is tested by the electron paramagnetic resonance (EPR) method to optimize the drug formulation. Then the loading factor of bound paclitaxel needs to be tested and optimized by high performance liquid chromatography (HPLC) analysis. Finally the size of the nanoparticle obtained by high pressure homogenization needs to be tested and optimized to 120 nm.

The formulation will be used MCFRN-HSA for its anti-proliferation, anti-progression and anti-metastasis cancer therapy to reduce mortality in cancer patients. The invention achieves its desired result because of its anti-hypoxic activity, its ability to restore blood flow, and its ability to simultaneous delivery of the anticancer drug paclitaxel into the hypoxic region of the cancer as a result of restoration of vascular NO. As a result of increased blood flow, MCFRN-HSA mediated the delivery of the drug paclitaxel or other anti-cancer chemotherapeutic drugs e.g. doxorubicin and may show equal or greater anti-mitotic efficacy without pro-tumorgenic cascade activities to that obtained using unmodified albumin or other drug carrier.

MCFRN-HSA would be used clinically alone or in combination with other therapies, such as chemotherapy, immunotherapy, surgery or radiation therapy to increase the survival of cancer patients.

### Anti-ROS Activities of MCFRN-HSA and Methods of Use

We present results to show that the inhibit the proliferation of TNBC breast cancer cell line 4T1 in vitro by MCFRN-HSA is dose dependent and is in the micro molar range. This anti-proliferation activity is also dose dependently related to the reduction of intracellular ROS of the 4T1 cell line. This anti-proliferation activity is also associated with down regulation of cyclin D1, a marker of cancer cell proliferation. We have also demonstrated that MCFRN-HSA is efficacious in inhibiting the proliferation of mEER and MLM3 cell lines of head neck squamous cell carcinomas (HNSCC) cancer (results not shown). These results, together support that MCFRN-HSA has broad anti-progression and anti-metastasis activities against cancers with solid tumor. The mechanism of action of MCFRN-HSA is through the reduction of their intracellular ROS of these cancer cells and track them down throughout the body fluid to inhibit metastasis. Another beneficial aspect of the present invention is that a NAT drug like MCFRN-HSA can be used as therapeutic agent for a broad range of cancer with solid tumor. Specifically, MCFRN-HSA's efficacy can be tested in TNBC patients, which is beyond the scope of Trastuzumab treatment of HER2 positive breast cancer patients.

Of particular additional utility in MCFRN-HSA for breast cancer therapy, is to focus on the treatment of the 10%-20% of the TNBC breast cancer patients because they don't have estrogen and progesterone receptors and also don't overexpress the HER2 protein. Most breast cancers associated with the gene BRCA1 are triple-negative which make the patient enrollment for clinical trials easily selected for MCFRN-HSA therapy.

The TNBC patients generally respond well to chemotherapy given after surgery. But the cancer tends to come back. So far, no targeted therapies have been developed to help prevent cancer returning in women with TNBC. MCFRN-HSA may be particularly beneficial for TNBC patients with recurrent metastasis.

However, MCFRN-HSA may have a much broader application than Trastuzumab for the treatment of breast cancer that are not HER2 positive. Trastuzumab application is specific and limited to the anti-proliferation level. Based on the data presented, the MCFRN-HSA therapeutic activities have been shown to extend beyond proliferation to cancer progression and metastasis to a broad range of cancers with solid tumor e.g. HNSCC. Therefore, MCFRN-HSA is an improvement for the cure for cancer. Based on the background of how a cancer cell proliferate, progress in the formation of a solid tumor and how it escapes from the original location of the tumor to metastasize to multiple new locations to form new tumors can all be treated comprehensively by MCFRN-HSA.

In addition to simultaneously modulating the three key carcinogenesis processes of cancer described above, another key aspect of the present invention is that the methods of treatment and compositions of the NAT agents in this case the long in vivo half-life and activities of MCFRN-HSA are strategically positioned and present in all intravascular and extravascular volume distributed by the cancer cells in the body. MCFRN-HSA also has the efficacy to create a free radical free vascular space to restore or improve the blood flow to the hypoxic core of the solid tumor either in the original sites or at new metastasized locations. Hypoxia induced Hypoxia-inducible factor 1-alpha, also known as HIF-1-alpha, leads to activation of and signaling through this pathway is known to control cancer metabolism, angiogenesis, proliferation and cell survival.

The novelty of MCFRN-HSA therapy is that the proliferation, progression and metastasis of cancer therapy can all be treated with a single therapeutic agent. This therapeutic approach is not fully addressed by the current standard cancer therapies. Of particular deficit of the current cancer therapies is the lack of appreciation of the compartmentalization of the ROS reactions from intracellular to extra-cellular verses intravascular verses extravascular spaces. Especially the lack of understanding that ROS is the driving force of the carcinogenesis. Therefore, we have employed a catalytic free radical nitroxide carried on an albumin to exert its extracellular therapeutic effects to cover the full cancer proliferation, progression and metastasis processes. MCFRN-HSA is designed to modulate ROS activities of the cancer cells in all compartments of the body fluid. The fact that ⅖th of the albumin is in the intravascular fluid and the remaining ⅗th of albumin is distributed in the extravascular fluid indicate that the presence of the MCFRN-HSA in all body fluid is responsible for the following three key aspects of this drug:
1. The ability of the HSA to inhibit the cancerous cell proliferation via the reduction of intracellular ROS is the key innovative therapeutic approach by being able to distinguish the difference in the role of ROS in cancerous cell vs normal cell proliferation. This observation is supported by the demonstration of the MCFRN-HSA activity in inhibiting the proliferation of cancer cell line in vitro is correlated with down regulation of cyclin D1 level. These in vitro data would reflect the inhibition of cell cycling which can be dose related to in vivo animal models by measuring the cyclin D1 as a molecular marker. Thus we have established the key foundation of the feasibility of this innovative approach in future clinical trials to ascertain how MCFRN-HSA can inhibit the cancer cells from establishing a new tumor site.
2. The inhibition of lung metastasis of flank breast tumor in vivo is a remarkable observation that the MCFRN-HSA, when given extravascularly is present along the passage of cancer cell to inhibit metastasis. What is even more remarkable is that the metastasis of breast cancer is enhanced by the treatment of a preferred chemotherapeutic agent, Doxorubicin, which actually increased the lung metastasis. The demonstration that the treatment of MCFRN-HSA reversed the lung metastasis induced by Doxorubicin would lead to the use of MCFRN-HSA as an adjunctive therapy in breast cancer clinical trials where the mortality rate of breast cancer may be reduced through the inhibition of metastasis, especially in triple-negative patients.
3. The reduction in lung metastasis observed above is clearly related to the data showing that MCFRN-HSA has direct effects on cancer cell proliferation and that this is associated with decreased cyclin D1 levels. Therefore, it is possible that cyclin D1 levels in tumors can serve as a marker for direct effects of MCFRN-HSA on tumor growth. MCFRN-HSA also has indirect effects on tumors by altering tumor blood flow. Increased blood flow is expected to reverse the hypoxia that is a hallmark in solid tumors. Hypoxia leads to activation of Hif1α and signaling through this pathway is known to control cancer metabolism, angiogenesis, proliferation, and cell survival. Furthermore, there is substantial evidence that hypoxia promotes cancer metastasis by influencing multiple steps in the process. Thus, Hif1α expression may serve as a marker of the indirect effects of MCFRN-HSA on tumor blood flow and as an indicator of the efficacy of the drug against metastasis.
4. MCFRN-HSA was shown to enhance the blood flow to the hypoxic core of solid flank TNBC model in a mouse suggested that the origin and driver of metastasis of tumor can be inhibited to improve the survival of cancer patients. Especially, the potential to improve survival of the cancer patient with multiple recurrence of metastasis due to ineffective standard cancer therapy like TNBC patients
5. MCFRN-HSA was shown to be efficacious in the inhibition of head neck cancer cells that is resistant to radiation and chemotherapy in vitro suggest that positioned MCFRN-HSA in all body fluid of the cancer patients can reduce the dose requirements and side effect of cancer radiation therapy (results not shown).
6. MCFRN-HSA administered through IP was show to have the efficacy in the treatment of mouse model of brain cancer across the blood brain barrier similar to that in TNBC mouse model (results not shown). Therefore, give the high cost of treating human brain tumor without significant survival benefit, in this invention we propose to demonstrate MCFRN-HSA can be used to prolong symptom free living in brain cancer patients with a continuous infusion pump for up to 5-7 years to deliver focal drug administration via IT into the cerebral spinal fluid for the treatment of the proliferation, progression and metastasis of brain and spinal cancer.
7. MCFRN-HSA was shown to reduce lung metastasis of a TNBC flank tumor caused by the standard breast cancer chemotherapy with Doxorubicin, which is also well known side effect. It is remarkable that IP MCFRN-HSA is able to intercept the metastasis of escaped 4T1 cells induced flank tumor caused by Doxorubicin to the lung. This preclinical efficacy data would suggest that conjunctive MCFRN-HSA therapy with standard chemotherapy may improve the patient's survival.

The Claims of this invention are based on the specific preclinical results described here. More importantly this invention describes the translation of well-established therapeutic mechanisms of MCFRN-HSA from stroke and SCD to cancer therapy is based the extensive preclinical model studies including the inhibition of the extra vascular matrix breakdown in hemorrhagic transformation to the enhancement of blood flow without oxidative stress for focal ischemia in the brain. Furthermore, the anti-inflammatory, anti-ROS therapeutic activities and mechanisms of MCFRN-HSA also apply in the inhibition of cancer carcinogenesis described in the current invention.

Metastases of tumors to multiple organs, rather than primary tumors, are responsible for 90% of the cancer deaths. To prevent these deaths, improved ways to treat metastatic disease are needed. Blood flow and other mechanical factors influence the escape of cancer cells to specific organs, whereas molecular interactions between the cancer cells and the new organ influence the probability that the cells will grow there. Inhibition of the growth of metastases in secondary sites offers a promising approach for cancer cure.

Compared to acute stroke, the carcinogenesis leading to metastasis represents a complex series of chronic events which comprehensively covering many functional capabilities of a cell. These include tumor cell migration/proliferation and invasion into the tissues surrounding the primary tumor location, their subsequent intravasation into the blood or lymph vessels, extravasation back through vasculature to distal organs, and finally colonization/proliferation and angiogenesis of a new location to produce a second tumor body.

A growing body of scientific evidence has revealed that cancer surgery can increase the risk of metastasis. Even though this contradicts conventional medical thinking, the facts are undeniable. Therefore, the impact of metastasis from surgical removal of the primary tumor has to be addressed. The rationale of surgery is straightforward—if you can get rid of the cancer by removing it from the body, then a cure can likely be achieved. Unfortunately, this approach does not take into account that following surgery, the cancer will frequently metastasize (spread to different organs). Quite often the metastatic recurrence is far more serious than the original tumor. In fact, for many cancers it is the metastatic recurrence, and not the primary tumor that ultimately proves to be fatal. This problem is most suitably addressed by the therapeutic mechanism of MCFRN-HSA.

MCFRN-HSA becomes strategically present in all extra cellular body fluids to track down isolated cancer cells that break away from the primary tumor. The following cellular events must be occurring in the presence of MCFRN-HSA, first the cancer cell must breach the connective tissue immediately surrounding the tumor. Once this occurs, the cancer cell enters a blood or lymphatic vessel. To gain entry, the cancer cell must secrete enzymes that degrade the basement membrane of the blood vessel which are subject to inhibition by MCFRN-HSA. This is vitally important for MCFRN-HSA to inhibit the metastatic cancer cell as it uses the bloodstream and the intermediate steps for transport to other vital organs of the body (i.e., the liver, brain, or lungs) where it can form a new deadly tumor.

To complete its voyage, the cancer cell must adhere to the lining of the blood vessel where it degrades through and exits the basement membrane of the blood vessel. Its final task is to burrow through the surrounding connective tissue to arrive at its final destination, the organ. Now the cancer cell can multiply and form a growing colony, serving as the foundation for a new metastatic cancer. Fortunately, MCFRN-HSA is capable of following the cancer cell metastasis each and every step along the way. Therefore, MCFRN-HSA therapy is an ideal strategy to protect against the increased risk of metastasis.

The known activity of MCFRN-HSA is its ability, through its nitroxide moieties, to dismutate superoxide and degrade hydrogen peroxide. In the vasculature, MCFRN-HSA reduces superoxide, preventing it from reacting with nitric oxide and leading to enhanced blood flow.

Therefore, in this invention we present results below to show that MCFRN-HSA would increase blood flow within tumors, leading to a reversal of tumor hypoxia. Hypoxia is a major factor in tumor metabolism, growth and metastasis (Rankin and Giaccia, 2016). Increased oxygenation of tumors is therefore expected to have significant consequences for tumorigenesis.

The 4T1 breast cancer model is a useful model for studying metastasis because it closely models human breast cancer and shows the same patterns of metastasis (Aslakson and Miller, 1992; Pulaski and Ostrand-Rosenberg, 1998; Yoneda et al., 2000). When injected into syngeneic mice primary 4T1 tumors grow rapidly and spontaneously metastasize to lung, bone, brain, and liver. We therefore used this model to examine the effects of MCFRN-HSA on growth and metastasis of 4T1 mammary tumors. Cells were injected subcutaneously in the flank and tumors were allowed to develop for 4 days. Then the animals were treated with MCFRN-HSA at 1 ml/Kg, 3 times per week. At end point, lungs were harvested from the animals and stained with India ink. Visible metastatic lung nodules were counted for each animal. MCFRN-HSA treated animals showed a robust decrease in lung metastatic nodules. In another experiment tumor bearing animals were treated with Doxorubicin in the presence or absence of MCFRN-HSA. Doxorubicin is commonly used to treat breast cancer patients. In the presence of Doxorubicin, MCFRN-HSA treated mice also showed a significant reduction in lung metastases. In these experiments there was no significant difference in the growth of primary tumors. However, the finding of reduced metastasis if highly significant because it is metastatic disease that leads to more than 90% or all cancer related deaths.

Diagrams, Figures and Results are included to illustrate these paradigm shifts and showcase the therapeutic potency and efficacy of MCFRN-HSA in inhibition of cancer cell proliferation, progression and metastasis in support the Claims of this patent application

In the following detailed description of Figures on MCFRN-HSA for use in cancer, we have presented pre-clinical in vitro and in vivo efficacy data in TNBC. Similar results from head and neck squamous cell carcinoma (HNSCC) cancer (results not presented). The results of the TNBC studies which support the discovery of a novel therapeutic principle is disclosed in this invention. The utility of this invention of such NAT to inhibit cancer metastasis may represent an improvement of “cure” for some of the human cancer patients with solid tumor. We describe below how the use of MCFRN-HSA in therapy of preclinical model of TNBC may lead to major improvements in survival and quality of life for cancer patients.

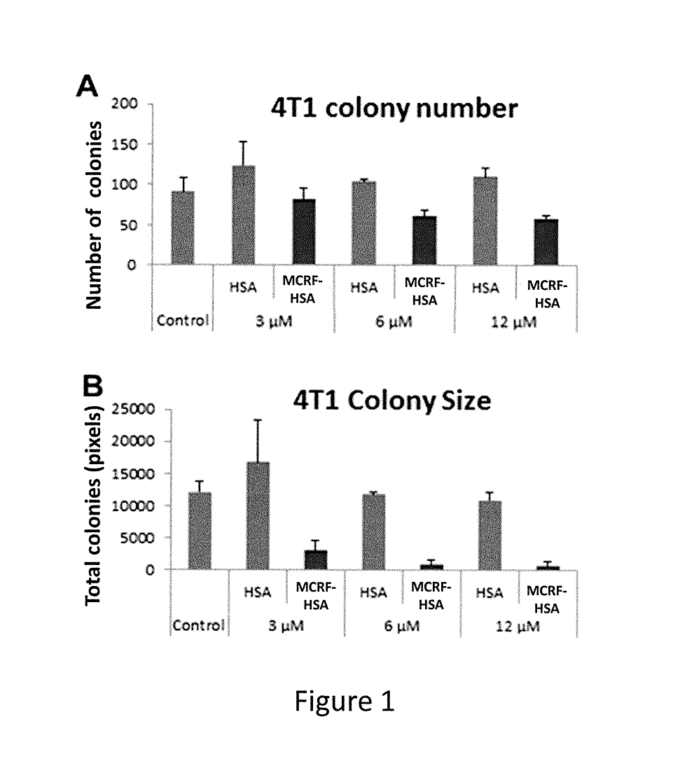

Reference is now made to FIG. 1: In Vitro studies to examine direct effects of MCFRN-HSA on cancer cells. These studies has been expand to test in vitro MCFRN-HSA concentrations across a broader dose range to define the optimize inhibition dose for inhibition of cancer cell proliferation. They have determined the inhibition effects of MCFRN-HSA on at least 3 cell lines derived from different cancer types and from normal tissues. We have studies the mechanisms by which MCFRN-HSA inhibits cancer cell proliferation by examining changes in intracellular levels of reactive oxygen species (ROS) in different cancerous cell lines and have examined the effects of MCFRN-HSA on cell cycle regulatory proteins.

It is known that cancer cells characteristically have higher levels of ROS than normal tissues (Montero and Jassem, 2011; Wondrak, 2009). This is because they frequently are deficient in antioxidant enzymes but produce ROS at higher rates than normal cells. These persistently elevated ROS levels activate signaling mechanisms that are able to promote cell proliferation and tumor progression. For example, it is known that ROS is able to activate phosphoinositol-3 kinase (PI3K) and MAPK signaling pathways which can lead to activation of Rac1 and the stimulation of cell proliferation (Behrend et al., 2003; Huang et al., 2013).

MCFRN-HSA possesses ROS catalytic breakdown activity and is able to eliminate superoxide and hydrogen peroxide, two key ROS produced by cells. Based on this information we have demonstrated that MCFRN-HSA directly inhibited on cancer cell proliferation. In this study, a TNBC modeling 4T1 breast cancer cells were plated at low density and incubated for 1 week to allow them to form colonies. The cells were treated with a range of MCFRN-HSA concentrations (FIG. 1) and control cells were left untreated or treated with the same concentrations of human serum albumin (HSA). Compared to HSA treated cells, MCFRN-HSA reduced the number of colonies formed, suggesting an effect on cell survival (FIG. 1a). More strikingly, MCFRN-HSA reduced colony size, suggesting that MCFRN-HSA blocks cell proliferation (FIG. 1b). These results have been confirmed by direct counting of cell number following MCFRN-HSA treatment (not shown). The effects of MCFRN-HSA on cell proliferation are observed at low micromolar concentrations of the drug.

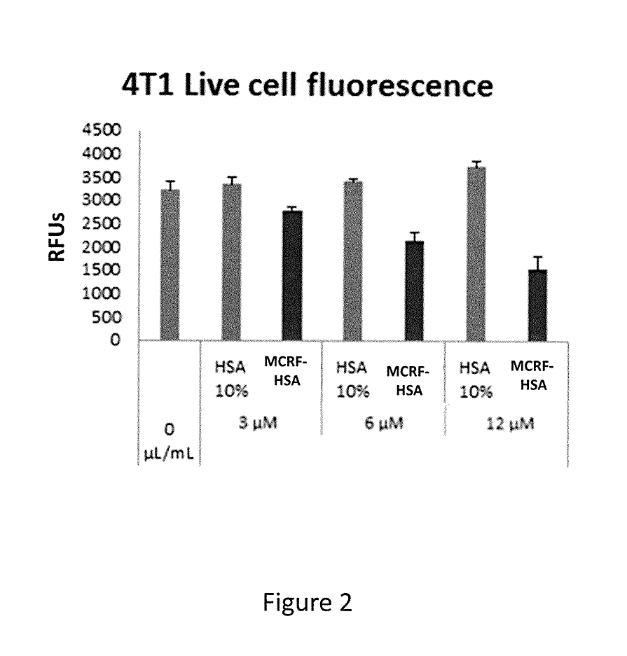

Reference is now made to FIG. 2. Since the known activity of MCFRN-HSA is the ability to act as a superoxide dismutase mimetic and catalytic agent for ROS removal, we examined its effects on cancer cell ROS levels. Cells were treated with different concentrations of MCFRN-HSA and then cellular ROS levels were estimated by staining with 2′,7′-dichlorodihydro-fluorescein diacetate (DCF) followed by flow cytometry (FIG. 2). The results demonstrate that MCFRN-HSA causes a dose dependent decrease in cellular ROS levels.

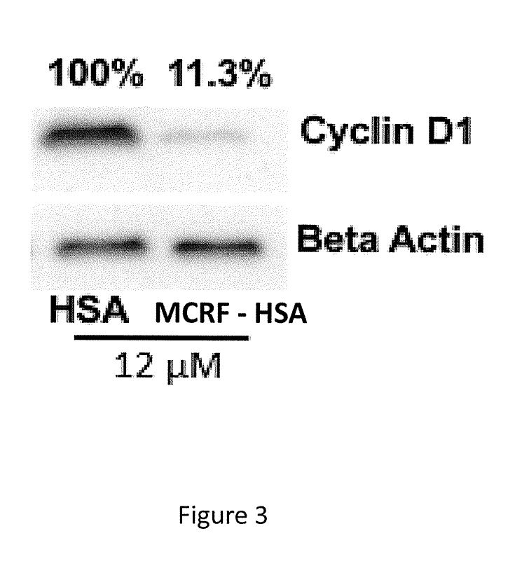

Reference is now made to FIG. 3. As an extension of our work showing that MCFRN-HSA inhibits cancer cell proliferation we examined a variety of cell cycle regulatory proteins. We found that cyclin D1 levels are decreased in MCFRN-HSA treated cancer cells (FIG. 3). This finding is important because cyclin D1 is a documented oncogene product that is commonly upregulated in human cancers and is considered a potential target for therapy (Musgrove et al., 2011). In summary, MCFRN-HSA appears to have direct effects on cancer cell proliferation. This is associated with reduced cellular ROS levels and the downregulation cyclin D1.

We have studied the MCFRN-HSA inhibition of proliferation in head and neck squamous cell carcinoma (HNSCC) cell lines, mEER and MLM3 and have obtained similar results as shown in that of FIG. 1-3 (not shown). MLM3 is of special interest because it is resistant to cisplatin and radiation therapy (CRT) and forms highly metastatic tumors in vivo. The cell lines were analyzed against the effect of CRT treatment, MCFRN-HSA was titrated across the same dose ranges and cell proliferation and survival determined by colony forming assays and direct cell counting as described above. Surprisingly, MCFRN-HSA's anti-proliferation efficacy was by far more superior than CRT (results not shown). Like TNBC, HNSCC has a high incidence rate with nearly 600,000 cases annually worldwide. The incidence of HPV+ HNSCC is increasing and is associated with severe comorbidities. About ˜10% of these cases are resistant to standard therapies, develop metastases, and is an incurable disease. Our results would indicate that MCFRN-HSA is such a potentially new and better treatments for this HNSCC patients.

These results are additional demonstrations that the inhibition of ROS with suppression of cyclin D1 expression is needed in addition to CRT treatment in order to fully inhibit cell proliferation in HNSCC patients.

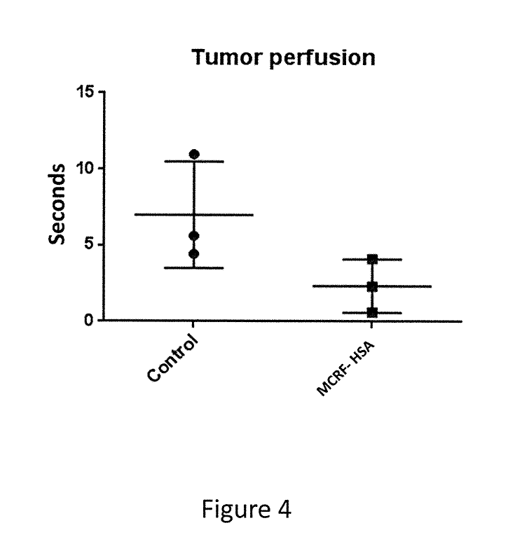

Reference is now made to FIG. 4: Measurement of blood flow in 4T1 tumors in mice. Tumor bearing animals were injected with microbubbles and then with HAS or MCFRN-HSA and time of tumor perfusion was measured by ultrasound. Lower time is indicative of faster blood flow through tumor.

MCFRN-HSA promotes blood flow in tumors. The known activity of MCFRN-HSA is its ability, through its nitroxide moieties, to dismutate superoxide and degrade hydrogen peroxide. In the vasculature, MCFRN-HSA reduces superoxide, preventing it from reacting with nitric oxide and leading to enhanced blood flow. In FIG. 4 we have demonstrated that MCFRN-HSA increase blood flow within tumors, leading to a reversal of tumor hypoxia. Hypoxia is a major factor in tumor metabolism, growth and metastasis (Rankin and Giaccia, 2016). Increased oxygenation of tumors is therefore expected to have significant consequences for tumorigenesis and metastasis.

Specifically, we have tested the effects of MCFRN-HSA on blood flow, Balb/c mice were injected with 4T1 breast cancer cells and tumors were allowed to develop for 8 days. Then tumor diffusion time was analyzed by ultrasound. For this, microbubbles were injected through the tail vein, and a baseline reading of tumor perfusion was obtained. This was followed by MCFRN-HSA or vehicle injection and a second reading of tumor perfusion time. Tumor perfusion was calculated by taking the 30 minute perfusion time minus the baseline time. A shorter time indicates greater perfusion of blood through the tumor. As shown in FIG. 4, the perfusion time for tumors in MCFRN-HSA treated animals was shorter than in vehicle treated animals. These results were confirmed by a second method using color Doppler ultrasound to image the tumor (not shown). Thus, as predicted MCFRN-HSA significantly increases blood flow to tumors.

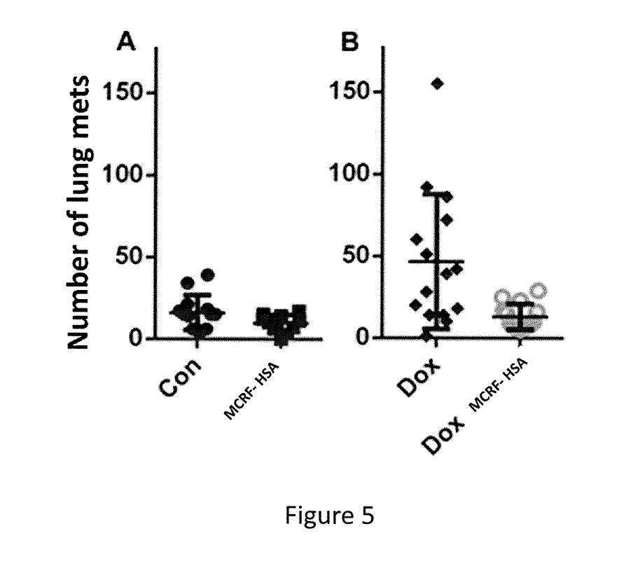

Reference is now made to FIG. 5. FIG. 5 shows the effect of MCFRN-HSA on lung metastasis in 4T1 breast cancer model. A) Tumor bearing mice were treated with or without MCFRN-HSA and after 3 weeks lungs were harvested for counting lung metastatic nodules. B) Same as A except that mice were also treated with doxorubicin. Metastatic nodules in the lung were significantly reduces with MCFRN-HSA treatment (P<0.01).

FIG. 5a shows that MCFRN-HSA inhibits metastasis in a mouse model of breast cancer. The 4T1 breast cancer model is a useful model for studying metastasis because it closely models human breast cancer and shows the same patterns of metastasis (Aslakson and Miller, 1992; Pulaski and Ostrand-Rosenberg, 1998; Yoneda et al., 2000). When injected into syngeneic mice primary 4T1 tumors grow rapidly and spontaneously metastasize to lung, bone, brain, and liver. We have used this model to examine the effects of MCFRN-HSA on growth and metastasis of 4T1 mammary tumors. Cells were injected subcutaneously in the flank and tumors were allowed to develop for 4 days. Then the animals were treated with MCFRN-HSA at 1 mg/Kg 3 times per week. At end point, lungs were harvested from the animals and stained with India ink. Visible metastatic lung nodules were counted for each animal (FIG. 5a). MCFRN-HSA treated animals showed a robust decrease in lung metastatic nodules. In another experiment tumor bearing animals were treated with Doxorubicin in the presence or absence of MCFRN-HSA. Doxorubicin is commonly used to treat breast cancer patients. In the presence of Doxorubicin, MCFRN-HSA treated mice also showed a significant reduction in lung metastases (FIG. 5b). In these experiments there was no significant difference in the growth of primary tumors. However, the finding of reduced metastasis if highly significant because it is metastatic disease that leads to more than 90% or all cancer related deaths.

The Disclosure of Examples of Preferred Compositions and Methods of their Use does not Limit this Invention to the Claims Below

### REFERENCES

1. Malorni L, Shetty P B, De Angelis C, Hilsenbeck S, Rimawi M F, Elledge R, Osborne C K, De Placido S, Arpino G. Clinical and biologic features of triple-negative breast cancers in a large cohort of patients with long-term follow-up. Breast Cancer Res Treat. 2012; 136(3):795-804. doi: 10.1007/s10549-012-2315-y. PubMed PMID: 23124476; PMCID: PMC3513514.

2. Chikarmane S A, Tirmani S H, Howard S A, Jagannathan J P, DiPiro P J. Metastatic patterns of breast cancer subtypes: what radiologists should know in the era of personalized cancer medicine. CLin Radiol, 2015; 70(1):1-10. Doi 10.1016/J.Crad 2014.08.015. PubMed PMID 25300558.

3. Maeda H. Toward a full understanding of the EPR effect in primary and metastatic tumors as well as issues related to its heterogeneity. Adv Drug Deliv Rev. 2015; 91:3-6. doi: 10.1016/j.addr.2015.01.002. PubMed PMID: 25579058.

4. Montero A J, Jassem J. Cellular redox pathways as a therapeutic target in the treatment of cancer. Drugs. 2011; 71(11):1385-96. doi: 10.2165/11592590-000000000-00000. PubMed PMID: 21812504.

5. Mustacchi G, De Laurentiis M. The role of taxanes in triple-negative breast cancer: literature review. Drug Des Devel Ther. 2015; 9:4303-18. doi: 10.2147/DDDT.S86105. PubMed PMID: 26273192; PMCID: PMC4532347.

6. Wondrak G T. Redox-directed cancer therapeutics: molecular mechanisms and opportunities. Antioxid Redox Signal. 2009; 11(12):3013-69. doi: 10.1089/ARS.2009.2541. PubMed PMID: 19496700; PMCID: PMC2824519.

7. Behrend L, Henderson G, Zwacka R M. Reactive oxygen species in oncogenic transformation. Biochem Soc Trans. 2003; 31(Pt 6):1441-4. doi: 10.1042/. PubMed PMID: 14641084.

8. Huang J S, Cho C Y, Hong C C, Yan M D, Hsieh M C, Lay J D, Lai G M, Cheng A L, Chuang S E. Oxidative stress enhances Axl-mediated cell migration through an Akt1/Rac1-dependent mechanism. Free Radic Biol Med. 2013; 65:1246-56. doi: 10.1016/j.freeradbiomed.2013.09.011. PubMed PMID: 24064382.

9. Musgrove E A, Caldon C E, Barraclough J, Stone A, Sutherland R L. Cyclin D as a therapeutic target in cancer. Nat Rev Cancer. 2011; 11(8):558-72. doi: 10.1038/nrc3090. PubMed PMID: 21734724.

10. Rankin E B, Giaccia A J. Hypoxic control of metastasis. Science. 2016; 352(6282):175-80. doi: 10.1126/science.aaf4405. PubMed PMID: 27124451; PMCID: PMC4898055.

11. Aslakson C J, Miller F R. Selective events in the metastatic process defined by analysis of the sequential dissemination of subpopulations of a mouse mammary tumor. Cancer Res. 1992; 52(6):1399-405. PubMed PMID: 1540948.

12. Pulaski B A, Ostrand-Rosenberg S. Reduction of established spontaneous mammary carcinoma metastases following immunotherapy with major histocompatibility complex class II and B7.1 cell-based tumor vaccines. Cancer Res. 1998; 58(7):1486-93. PubMed PMID: 9537252.

13. Yoneda T, Michigami T, Yi B, Williams P J, Niewolna M, Hiraga T. Actions of bisphosphonate on bone metastasis in animal models of breast carcinoma. Cancer. 2000; 88(12 Suppl):2979-88. PubMed PMID: 10898341.

14. Vyas D, Laput G, Vyas A K. Chemotherapy-enhanced inflammation may lead to the failure of therapy and metastasis. Onco Targets Ther. 2014; 7:1015-23. doi: 10.2147/OTT.S60114. PubMed PMID: 24959088; PMCID: PMC4061164.

15. Spano D, Heck C, De Antonellis P, Christofori G, Zollo M. Molecular networks that regulate cancer metastasis. Semin Cancer Biol. 2012; 22(3):234-49. doi: 10.1016/j.semcancer.2012.03.006. PubMed PMID: 22484561.

16. Haugrud A B, Zhuang Y, Coppock J D, Miskimins W K. Dichloroacetate enhances apoptotic cell death via oxidative damage and attenuates lactate production in metformin-treated breast cancer cells. Breast Cancer Res Treat. 2014. doi: 10.1007/s10549-014-3128-y. PubMed PMID: 25212175.

17. Zhuang Y, Miskimins W K. Cell cycle arrest in Metformin treated breast cancer cells involves activation of AMPK, downregulation of cyclin D1, and requires p27Kip1 or p21Cip1. J Mol Signal. 2008; 3:18. Epub 2008 Dec. 3. doi: 1750-2187-3-18 [pii] 10.1186/1750-2187-3-18. PubMed PMID: 19046439; PMCID: 2613390.

18. Zhuang Y, Miskimins W K. Metformin Induces Both Caspase-Dependent and Poly(ADP-ribose) Polymerase-Dependent Cell Death in Breast Cancer Cells. Mol Cancer Res. 2011; 9:603-15. Epub 2011 Mar. 23. doi: 1541-7786.MCR-10-0343 [pii] 10.1158/1541-7786.MCR-10-0343. PubMed PMID: 21422199.

19. Coppock J D, Vermeer P D, Vermeer D W, Lee K M, Miskimins W K, Spanos W C, Lee J H. mTOR inhibition as an adjuvant therapy in a metastatic model of HPV+ HNSCC. Oncotarget. 2016. doi: 10.18632/oncotarget.8286. PubMed PMID: 27015118.

20. Fuste N P, Fernandez-Hernandez R, Cemeli T, Mirantes C, Pedraza N, Rafel M, Torres-Rosell J, Colomina N, Ferrezuelo F, Dolcet X, Gari E. Cytoplasmic cyclin D1 regulates cell invasion and metastasis through the phosphorylation of paxillin. Nature communications. 2016; 7:11581. doi: 10.1038/ncomms11581. PubMed PMID: 27181366; PMCID: PMC4873647.

21. Lehn S, Tobin N P, Berglund P, Nilsson K, Sims A H, Jirstrom K, Harkonen P, Lamb R, Landberg G. Down-regulation of the oncogene cyclin D1 increases migratory capacity in breast cancer and is linked to unfavorable prognostic features. Am J Pathol. 2010; 177(6):2886-97. doi: 10.2353/ajpath.2010.100303. PubMed PMID: 20971731; PMCID: PMC2993304.

I claim:

1. A nitroxylated macromolecule to inhibit cancer in cancer patients comprising:
a. a macromolecule possessing anti-reactive oxygen species (ROS) activity;
b. a macromolecule that enhances blood flow;
c. a nitroxide that has a piperidine, pyrolidine or pyrolline ring; and
d. a macromolecule that is serum albumin or other biocompatible colloid.

2. The nitroxylated macromolecule of claim 1, wherein the nitroxylated macromolecule inhibits one or more of the following cancer stages:
a. cancer cell proliferation;
b. cancer progression;
c. cancer metastasis;
d. morbidity due to cancer;
e. morbidity due to other cancer treatment;
f. mortality due to cancer; or
g. mortality due to other cancer treatments.

3. The nitroxylated macromolecule of claim 1, wherein the nitroxylated macromolecule is administered into the body by means of one or more of the following administration methods:
a. intravenous;
b. intraperitoneal;
c. intraosseus;
d. epidural;
e. intracerebral;
f. intracerebroventricular;
g. intraportal;
h. intradermal;
i. subcutaneous;
j. intramuscular;
k. intrathecal;
l. intraportal;
m. intravitreal;
n. intraarticular; or
o. cerebrospinal infusion.

4. A nitroxylated macromolecule that following administration exerts anti-cancer effects in a body compartment into which it was not directly administered, including:
a. vascular system;
b. cerebrospinal fluid;
c. lymphatic system;
d. skin;
e. central and peripheral nervous system; or
f. any organ.

5. The method of treatment of cancer comprising administration of a nitroxylated macromolecule as the only therapy or in combination with another drug or therapy.

6. The nitroxylated macromolecule of claim 1, wherein the nitroxylated macromolecule localizes in all needed body fluid compartments for use in treatment of cancer carcinogenesis leading to the inhibition of metastasis of cancer at one or more locations in the body of the patient.

7. The nitroxylated macromolecule of claim 1, wherein the nitroxylated macromolecule is for use as a blood flow enhancer or restorer of oxygen delivery to the hypoxic core of one or more tumors to inhibit carcinogenesis.

8. The nitroxylated macromolecule of claim 1, wherein the nitroxylated macromolecule is for use as an inhibitor of cancer cell proliferation and progression through the reduction of the cellular reactive oxygen species levels in cancer cells and for conjunctive use with standard cancer therapeutic procedures to improve cancer patient survival.

9. The nitroxylated macromolecule of claim 1, wherein the nitroxylated macromolecule is for use in the provision of inhibition of metastasis induced by conventional cancer therapies to reduce the mortality of cancer patients.

10. The nitroxylated macromolecule of claim 1, wherein the nitroxylated macromolecule is for use for the inhibition of target molecules which are responsible for cancer cell division which results in proliferation and progression of the cancer.

11. The nitroxylated macromolecule of claim 1, wherein the nitroxylated macromolecule is positioned in all body fluids of the cancer patients to intercept escaped cancer cells and inhibit proliferation, progression or metastasis.

12. The nitroxylated macromolecule of claim 1, wherein the nitroxylated macromolecule is used as a carrier to enhance the therapeutic index of a lipophilic cancer chemotherapeutic agent.

13. The nitroxylated macromolecule of claim 1, wherein the nitroxylated macromolecule is positioned in all body fluid of the cancer patients to reduce the side effects of cancer radiation therapy.

14. The nitroxylated macromolecule of claim 1, wherein the nitroxylated macromolecule is used to improve survival of the cancer patient with multiple recurrence of metastasis.

15. The nitroxylated macromolecule of claim 1, wherein the nitroxylated macromolecule can be locally administered into the cerebral spinal fluid for the treatment of the proliferation, progression and metastasis of brain or spinal cancer.

16. The nitroxylated macromolecule of claim 1, wherein the nitroxylated macromolecule is positioned in all body fluids of the cancer patients to intercept cancer cells escaped from the primary tumor to inhibit their proliferation, progression and metastasis to secondary sites caused by standard chemotherapy.

17. The nitroxylated macromolecule of claim 1, wherein the nitroxylated macromolecule is positioned in a plurality of body fluids of the cancer patient to intercept escaped cancer cells and inhibit proliferation, progression, or metastasis.
